(19) United States
(12) Patent Application Publication
(10) Pub. No.: US 2018/0128752 A1
(43) Pub. Date: May 10, 2018

(54) Visual Inspection of Difficult to Inspect Fluids

(71) Applicant: Sandoz Canada Inc., Boucherville (CA)

(72) Inventors: Louis-Philippe Labranche, Laval (CA); Éric Vincent, Boucherville (CA); Yves Leblanc, Montréal (CA)

(73) Assignee: Sandoz Canada Inc., Boucherville (CA)

(21) Appl. No.: 15/805,246

(22) Filed: Nov. 7, 2017

(30) Foreign Application Priority Data
Nov. 7, 2016 (CA) 2947806

(51) Int. Cl. G01N 21/94; G01N 1/34; G01N 21/88; G01N 33/15

(52) U.S. Cl. CPC G01N 21/94; G01N 33/15; G01N 21/8803; G01N 1/34

(57) ABSTRACT

The present invention relates to novel methods for detection of the presence or absence of particulate matter in an opaque fluid, novel methods for inspection of an opaque fluid for the presence or absence of particulate matter therein, and use of such methods in the manufacture of an opaque fluid. Methods comprise clarifying the fluid to enable detection of the presence or absence of particulate matter therein or to enable inspection of the opaque fluid for the presence or absence of particulate matter therein.

### CROSS REFERENCE TO RELATED APPLICATIONS

The present patent application claims benefit to Canadian patent application number 2,947,806 filed Nov. 7, 2016, entitled VISUAL INSPECTION OF DIFFICULT TO INSPECT FLUIDS, by Louis-Philippe Labranche, Eric Vincent and Yves Leblanc, docket number PAT057416, and which is hereby incorporated by reference herein in its entirety.

### FIELD OF THE INVENTION

The present invention relates to methods for detection of particulate matter in an opaque fluid. More particularly, the present invention relates to methods for detection of particulate matter in an opaque fluid to be used or consumed by or administered to an animal. The present invention also relates to methods for inspection of an opaque fluid for the presence or absence of particulate matter. The present invention further relates to methods for inspection of an opaque fluid to be used or consumed by or administered to an animal for the presence or absence of particulate matter.

### BACKGROUND OF THE INVENTION

Methods and technologies for inspection of fluids for the presence or absence of particulate matter are known and include, for example, the use of light obscuration (as in Light Obscuration Particle Count Test), microscopic inspection (as in Microscopic Particle Count Test) and rotary spin methods. Specific methods for the determination of particulate matter in certain fluids are described, for example, in the appropriate sections of the U.S. Pharmacopeia.

For clear fluids, visual inspection of an entire manufactured volume or of smaller portioned or packaged volumes in individual container units can be relatively easily performed either by automated or manual inspection. However, where the nature of the fluid permits only limited or no capacity for visual inspection, such as when the fluid is opaque, visual inspection of both the entire volume or of smaller portioned or packaged volumes in individual container units is much more difficult.

In the pharmaceutical, cosmetic and beverage industries, for example, finished product contained in bottles, bags, vials, ampoules, syringes and the like, needs to be visually inspected to determine the presence or absence of particulate matter before it can be considered safe for use or consumption. In particular, containers such as bottles, bags, vials, ampoules, syringes and the like containing injectable or ophthalmic solutions, for example, require that a comprehensive visual inspection be performed by qualified inspectors.

Fluids that are colorless, transparent or translucent easily facilitate inspection. In specific cases, however, fluids to be inspected may be opaque. For such fluids, visual inspection becomes more difficult to do because it becomes difficult to distinguish between particulate matter and the fluid itself. In other cases, the product to be inspected is a dry solid product, such as a lyophilisate, which does not lend itself to efficient inspection for the presence or absence of particulate matter.

There is thus a need for new methods for distinguishing between particulate matter in an opaque fluid and the opaque fluid itself. There is also a need for new methods for detection of particulate matter in an opaque fluid, such as an opaque fluid to be used or consumed by or administered to an animal. There is also a need for new methods for inspection of an opaque fluid for the presence or absence of particulate matter, such as an opaque fluid to be used or consumed by or administered to an animal. There is also a need for new methods for distinguishing between particulate matter in a dry solid product and the dry solid product itself. There is also a need for new methods for detection of particulate matter in a dry solid product, such as in a lyophilized product within a container. There is also a need for new methods for inspection of a dry solid product, such as a lyophilized product within a container, for the presence or absence of particulate matter.

### SUMMARY OF THE INVENTION

The present invention relates to new methods for distinguishing between particulate matter in an opaque fluid and the opaque fluid itself. The present invention also relates to methods for detection of particulate matter in an opaque fluid, such as an opaque fluid to be used or consumed by or administered to an animal. The present invention also relates to methods for inspection of an opaque fluid for the presence or absence of particulate matter, such as an opaque fluid to be used or consumed by or administered to an animal. The present invention also relates to new methods for distinguishing between particulate matter in a dry solid product and the dry solid product itself. The present invention further relates to methods for detection of particulate matter in a dry solid product, such as in a lyophilized product within a container, and to methods for inspection of a dry solid product, such as a lyophilized product within a container, for the presence or absence of particulate matter.

Accordingly, in an embodiment of the invention, the invention relates to a method for distinguishing between particulate matter in an opaque fluid and the opaque fluid itself, the method comprising removing a component of the opaque fluid which causes the fluid to be opaque.

In another embodiment of the invention, the invention relates to a method of clarifying an opaque fluid to enable detection of the presence or absence of particulate matter in the fluid, the method comprising removing a component of the opaque fluid which causes the fluid to be opaque, thereby clarifying the fluid.

In another embodiment of the invention, the invention relates to a method of clarifying an opaque fluid to enable inspection of the fluid for the presence or absence of particulate matter in the fluid, the method comprising removing a component of the opaque fluid which causes the fluid to be opaque, thereby clarifying the fluid.

In another embodiment of the invention, the invention relates to a method of clarifying an opaque fluid to enable identification of particulate matter in the fluid, the method comprising removing a component of the opaque fluid which causes the fluid to be opaque, thereby clarifying the fluid.

In another embodiment of the invention, the invention relates to a method of clarifying an opaque fluid to enable visualization of the presence or absence of particulate matter in the fluid, the method comprising removing a component of the opaque fluid which causes the fluid to be opaque, thereby clarifying the fluid.

In another embodiment of the invention, the invention relates to use of a method of clarifying an opaque fluid to enable detection of the presence or absence of particulate matter in the fluid.

In another embodiment of the invention, the invention relates to use of a method of clarifying an opaque fluid to enable inspection of the fluid for the presence or absence of particulate matter in the fluid.

In another embodiment of the invention, the invention relates to use of a method of clarifying an opaque fluid to enable identification of particulate matter in the fluid.

In another embodiment of the invention, the invention relates to use of a method of clarifying an opaque fluid to enable visualization of the presence or absence of particulate matter in the fluid.

In another embodiment of the invention, the invention relates to a method for detection of the presence or absence of particulate matter in an opaque fluid, the method comprising clarifying the fluid to enable detection of the presence or absence of particulate matter therein.

In another embodiment of the invention, the invention relates to a method for inspection of an opaque fluid for the presence or absence of particulate matter, the method comprising clarifying the fluid to enable the inspection of the fluid for the presence or absence of particulate contamination of the fluid.

In another embodiment of the invention, the invention relates to a method for identification of particulate matter in an opaque fluid, the method comprising clarifying the fluid to enable the identification of the particulate matter therein.

In another embodiment of the invention, the invention relates to a method of visualizing the presence or absence of particulate matter in an opaque fluid, the method comprising clarifying the fluid, wherein clarifying the fluid enables visualization of the particulate matter therein.

In another embodiment of the invention, the invention relates to a device for detecting the presence or absence of particulate matter in an opaque fluid, the device comprising means for clarifying the fluid to enable detection of the presence or absence of particulate matter therein.

In another embodiment of the invention, the invention relates to a device or inspection of an opaque fluid for the presence or absence of particulate matter, the device comprising means for clarifying the fluid to enable inspection of the fluid for the presence or absence of particulate matter therein.

In another embodiment of the invention, the invention relates to a device for identification of particulate matter in an opaque fluid, the device comprising means for clarifying the fluid to enable identification of the particulate matter therein.

In another embodiment of the invention, the invention relates to a device for visualizing the presence or absence of particulate matter in an opaque fluid, the device comprising means for clarifying the fluid to enable visualizing the particulate matter therein.

In another embodiment of the invention, the invention relates to a method, use of a method and/or device described herein in a quality control process for release of a quantity of opaque fluid during or after manufacture of the fluid.

In another embodiment of the invention, the invention relates to a quality control process for release of a quantity of opaque fluid during or after manufacture of the fluid, comprising a use, a method, use of a method and/or device described herein.

In certain embodiments, clarifying the fluid comprises removing a component of the opaque fluid which causes the fluid to be opaque, thereby clarifying the fluid.

The foregoing merely summarizes certain embodiments of the invention and is not intended to be limiting in nature. These and other embodiments of the invention will become apparent from the following description of other embodiments. The detailed description is merely illustrative of the invention and does not limit the scope of the invention, which is defined by the appended claims and equivalents thereof. As would be obvious to one skilled in the art, many variations and modifications of the invention may be effected without departing from the spirit and scope of the novel concepts of the disclosure.

### BRIEF DESCRIPTION OF THE FIGURES

Not Applicable.

### DETAILED DESCRIPTION

The following description of embodiments of the invention is for illustrative purposes only and is not intended to limit the scope of the invention.

The patent and scientific literature referred to herein establishes knowledge that is available to those with skill in the art. Any issued patents, applications, and references that are cited herein are hereby incorporated by reference to the same extent as if each was specifically and individually indicated to be incorporated by reference. In the case of inconsistencies, the present disclosure will prevail.

Agents that may clarify an opaque fluid are known in the chemical art. Some non-limiting examples include the addition of concentrated HCl to an iron oxide solution (Fe III) to transform the iron oxide solution (dark red opaque solution) to an iron chloride (Fe II) solution (light orange solution), and colour changes of dyes and indicators in response to solution pH. It is also known in the chemical art that certain oxidizing agents bleach materials (i.e. reduce colour) by partial or complete degradation of colorant molecules. U.S. Pat. No. 4,196,017, for example, describes a method for reducing colour impurities in sugar-containing syrups using hydrogen peroxide.

However, the application of clarifying agents for distinguishing between particulate matter in an opaque fluid and the opaque fluid itself has not been reported. In particular, the application of such agents, to enable the inspection of an opaque fluid for the presence or absence of particulate matter or to enable the detection, identification or visualization of particulate matter in an opaque fluid has not been reported. In particular the application of such agents in a quality control process for an opaque fluid, to enable the inspection of the fluid for the presence or absence of particulate matter or to enable the detection, identification or visualization of particulate matter in the fluid has not been reported. In particular, the application of such agents in a quality control process for an opaque pharmaceutical, cosmetic or beverage to enable the inspection of the pharmaceutical, cosmetic or beverage for the presence or absence of particulate matter or to enable the detection, identification or visualization of particulate matter in the pharmaceutical, cosmetic or beverage has not been reported. In particular, the application of such agents in a quality control process for an opaque pharmaceutical product (non-limiting example is an injectable product such as an iron containing product, such as an iron dextran product or a ferric carboxymaltose product) to enable the inspection of the pharmaceutical product for the presence or absence of particulate matter or to enable the detection, identification or visualization of particulate matter in the pharmaceutical product has not been reported. In particular, the application of clarifying agents for distinguishing between particulate matter in a dry solid product and the dry solid product itself has not been reported.

Use of the term “application of such agents” is intended to include, but not be limited to, use of such agents, methods of using such agents, methods comprising such agents, processes comprising such agents, devices using such agents and devices comprising means to implement such agents.

It has now been surprisingly discovered that clarifying an opaque fluid (i.e., decreasing or removing the opaqueness of an opaque fluid (alternatively, increasing the transparency of an opaque fluid)) increases the contrast between particulate matter in the fluid and the fluid itself and thus increases the ability to distinguish between particulate matter in the fluid and the fluid itself. It has also been surprisingly discovered that the use of a clarifying agent increases the contrast between particulate matter in an opaque fluid and the fluid itself and thus increases the ability to distinguish between particulate matter in an opaque fluid and the fluid itself. It has also been surprisingly discovered that the application of such agents may enable the inspection of an opaque fluid for the presence or absence of particulate matter or may enable the detection, identification or visualization of particulate matter in an opaque fluid. In particular, it has now been surprisingly discovered that the application of such agents in a quality control process for an opaque fluid, may enable the inspection of the fluid for the presence or absence of particulate matter or may enable the detection, identification or visualization of particulate matter in the fluid. In particular, it has also been surprisingly discovered that the application of such agents in a quality control process for an opaque pharmaceutical, cosmetic or beverage, may enable the inspection of the pharmaceutical, cosmetic or beverage for the presence or absence of particulate matter or may enable the detection, identification or visualization of particulate matter in the pharmaceutical, cosmetic or beverage. In particular, it has also been surprisingly discovered that the application of such agents in a quality control process for an opaque pharmaceutical product (non-limiting example is an injectable product, such as an iron containing product, such as an iron dextran product or a ferric carboxymaltose product) may enable the inspection of the pharmaceutical product for the presence or absence of particulate matter or may enable the detection, identification or visualization of particulate matter in the pharmaceutical product.

In particular, it has been surprisingly discovered that clarifying an opaque fluid may enable the inspection of the fluid for the presence or absence of particulate matter or may enable the detection, identification or visualization of particulate matter in the fluid. In particular, it has also been surprisingly discovered that in a quality control process for an opaque fluid, clarifying the fluid may enable the inspection of the fluid for the presence or absence of particulate matter or may enable the detection, identification or visualization particulate matter in the fluid. In particular, it has also been surprisingly discovered that in a quality control process for an opaque pharmaceutical, cosmetic or beverage, clarifying the opaque pharmaceutical, cosmetic or beverage may enable the inspection of the pharmaceutical, cosmetic or beverage for the presence or absence of particulate matter or may enable the detection, identification or visualization of particulate matter in the pharmaceutical, cosmetic or beverage. In particular, it has also been surprisingly discovered that in a quality control process for an opaque pharmaceutical product (non-limiting example is an injectable product, such as an iron containing product, such as an iron dextran product or a ferric carboxymaltose product), clarifying the opaque pharmaceutical product may enable the inspection of the pharmaceutical product for the presence or absence of particulate matter or may enable the detection, identification or visualization of particulate matter in the pharmaceutical product.

The inventions disclosed herein may therefore be useful in, or as part of, a quality control method or process used for regulatory approval of an opaque fluid by a governmental agency, or for the clearance or release of a manufactured opaque fluid, for example, for sale to a consumer or use by an animal.

Accordingly, in certain embodiments of the invention, the invention relates to a method for distinguishing between particulate matter in an opaque fluid and the opaque fluid itself, the method comprising removing a component of the opaque fluid which causes the fluid to be opaque.

In another embodiment, of the invention, the invention relates to a method of clarifying an opaque fluid, or use of such method, to enable detection or visualization of the presence or absence of particulate matter in the fluid, the method comprising removing a component of the opaque fluid which causes the fluid to be opaque, thereby clarifying the fluid.

In another embodiment of the invention, the invention relates to a method of clarifying an opaque fluid, or use of such method, to enable inspection of the fluid for the presence or absence of particulate matter in the fluid, the method comprising removing a component of the opaque fluid which causes the fluid to be opaque, thereby clarifying the fluid.

In another embodiment of the invention, the invention relates to a method of clarifying an opaque fluid, or use of such method, to enable identification of particulate matter in the fluid, the method comprising removing a component of the opaque fluid which causes the fluid to be opaque, thereby clarifying the fluid.

In another embodiment of the invention, the invention relates to a method for detection of the presence or absence of particulate matter in an opaque fluid, the method comprising clarifying the fluid to enable detection of the presence or absence of particulate matter therein.

In another embodiment of the invention, the invention relates to a method for visualization of the presence or absence of particulate matter in an opaque fluid, the method comprising clarifying the fluid to enable visualization of the presence or absence of particulate matter therein.

In another embodiment of the invention, the invention relates to method for inspection of an opaque fluid for the presence or absence of particulate matter, the method comprising clarifying the fluid to enable the inspection of the fluid for the presence or absence of particulate contamination of the fluid.

In another embodiment of the invention, the invention relates to a method for identification of a particulate matter in an opaque fluid, the method comprising clarifying the fluid to enable the identification of the particulate matter therein.

For the purpose of the present disclosure, certain terms are defined below.

The terms “clarify”, “clarifying”, “clarification” and the like are intended to mean to make a fluid more transparent. In certain embodiments of the present invention, clarifying a fluid comprises removing all opaqueness such that the fluid is 100% transparent. In certain embodiments of the present invention, clarifying a fluid comprises reducing opaqueness or increasing transparency of the fluid to a sufficient degree to enable distinguishing between particulate matter in the fluid and the fluid itself. In certain embodiments, of the present invention, clarifying a fluid comprises reducing opaqueness or increasing transparency of the fluid to a sufficient degree to enable the detection, identification or visualization of particulate matter therein. In certain embodiments of the present invention, clarifying a fluid comprises reducing opaqueness or increasing transparency of the fluid to a sufficient degree to enable inspection of the fluid for the presence or absence of particulate matter therein. In certain embodiments, clarifying an opaque fluid comprises removing component of the fluid which causes the fluid to be opaque.

The term “transparent” is intended to mean that a fluid transmits incident light. In certain embodiments of the invention, a clarified fluid transmits at least 50% of incident light. In certain embodiments of the invention, a clarified fluid transmits at least 80% of incident light. In certain embodiments of the invention, a clarified fluid transmits at least 90% of incident light. In certain embodiments of the invention, a clarified fluid transmits at least 95% of incident light. In certain embodiments of the invention, a clarified fluid transmits at least 99% of incident light, or 100% of incident light. In certain embodiments of the present invention, a clarified fluid transmits sufficient incident light to enable distinguishing between particulate matter in the fluid and the fluid itself. In certain embodiments of the invention, a clarified fluid transmits sufficient incident light to enable inspection of the fluid for the presence or absence of particulate matter therein. In certain embodiments of the invention, a clarified fluid transmits sufficient incident light to enable the detection, identification or visualization of particulate matter therein.

The term “opaque fluid” is intended to mean that a fluid that does not transmit sufficient incident light to enable distinguishing between particulate matter in the fluid and the fluid itself. In certain embodiments of the invention, the fluid thus does not transmit sufficient incident light to enable inspection of the fluid for the presence or absence of particulate matter therein, or sufficient incident light to enable the detection identification or visualization of particulate matter therein.

The term “fluid” is intended to mean a flowable material. In certain embodiments the fluid is selected from the group consisting of solutions, suspensions, emulsions, syrups, gels, creams, pastes, and the like. In certain embodiments the fluid is a solution, emulsion or a suspension. In certain embodiments, the fluid is a solution. In certain embodiments, the fluid is a colloidal solution. In certain embodiments the fluid is a suspension. In certain embodiments, the is emulsion. In certain embodiments, the fluid is for pharmaceutical use. In certain embodiments, the fluids is for cosmetic use. In certain embodiments, the fluid is for consumption, such as a pharmaceutical product for oral administration. In certain embodiments, the fluid is for injection. In certain embodiments, the fluid is a beverage.

The term “beverage” as used herein is intended to mean any drinkable fluid suitable for consumption by a human or other animal. In certain embodiments the beverage is a liquid food such as, but not limited to, a liquid nutritional supplement, a liquid meal replacement or supplement, or a yogurt or yogurt-type drink. In certain embodiments, the beverage is selected from the group consisting of a soft drink (such as a cola), sport drink, energy drink, milk, juice, wine beer, or other alcohol-containing beverage, a coffee or coffee-based drink, and a tea or tea-based drink. In certain embodiments, the beverage is a cola. In certain embodiments, the beverage is drink. In certain embodiments, the beverage is beer.

In certain embodiments, the fluid comprises an iron compound. In certain embodiments the iron compound is an iron carbohydrate complex. In certain embodiments the iron compound is an iron dextran or an iron carboxymaltose, such as a ferric carboxymaltose. In certain embodiments, the iron compound is an iron oxide, such as Fe(III)OOH. In certain embodiments the fluid is a product such as CosmoFer®, INFeD® or Injectafer®. In certain embodiments the fluid is CosmoFe®, INFeD® Injectafer®.

The term “particulate matter” as used herein is intended to mean an extraneous mobile undissolved particle, other than a gas bubble, unintentionally present in a fluid. It is intended to encompass any undesirable non-solubilized matter in a fluid, whether it be naturally occurring in the fluid due to the properties of the fluid or the process to make it, or foreign in the sense that it did not originate from the fluid itself, and which is in the form of a discrete unit, particle, granule, or the like. Examples of such particulate matter include, but are not limited to, fiber, hair, glass, plastic, metal, elastomeric material, and precipitate.

The presence of particulate matter in a fluid may indicate that the fluid is contaminated, potentially non-sterile, and/or potentially unsafe for use, consumption or administration and is an important factor in determining whether the fluid will pass governmental and/or internal quality control requirements. In a multistep manufacturing process, for example, not passing a quality control requirement at one step can inhibit the release of the product for a subsequent manufacturing step, or alternatively inhibit the release of the final fluid product to consumers. Similarly, not passing a governmental quality control requirement can inhibit the regulatory approval of a fluid product. Either event can prevent or delay the release of the fluid product for sale to or use by a consumer. Alternatively, the presence of particulate matter in a fluid batch or lot previously released to the public for sale, consumption or administration may result in recall of one or more released batches or lots.

The particulate matter may be any one or more of an extrinsic particle, an intrinsic particle or an inherent particle.

The term “extrinsic particle” is intended to mean a particle that is not the result of part of the formulation, packaging or manufacturing process for the fluid. The origin of this type of particle is difficult to assess because happens randomly in a manufacturing process, but may come from sources such as, but not limited to, human, animal, insect (including arachnid and other insect-like source) or cellulose.

The term “intrinsic particle” is intended to mean a particle arising from sources related to formulation, packaging or manufacturing the fluid. It may come from different sources, such as, but not limited to, pieces of equipment, metal, silicone, rubber, plastic, glass (for example, broken bottle, vial, ampoule, etc.), blister, labeling, stopper, seal or other packaging and the like. The origin of this type of particle is easier to evaluate as it happens in a known manner in a manufacturing process.

The term “inherent particle” is intended to mean a particle which is directly connected to the fluid. It may come from different sources such as, but not limited to, oxidation of a raw material or an excipient used in the manufacture of the fluid, micelle formation, precipitation of an active ingredient (for example, polymorph or salt), or an excipient, or phase separation. The origin of this type of particle must be considered in the development phase to understand the nature of this type of particle and its impact on the product.

Visual inspection of a difficult to inspect fluid requires finding a way to distinguish between particulate matter in the fluid and the fluid itself, or to make any particulate matter in the fluid more visible. Different fluids will necessarily have different preparation methods for doing this.

The phrase “removing a component of the opaque fluid which causes the fluid to be opaque” is intended to mean eliminating or modifying one or more component of the opaque fluid such that the component no longer makes the fluid opaque. In certain embodiments, this comprises treating the opaque fluid with an appropriate clarifying agent selected to eliminate or modify such component of the opaque fluid such that the component no longer makes the fluid opaque. Non-limiting examples include agents that cause adjusting the dissociation of hydrogen ions from a component by changing the fluid pH such that the component changes colour, oxidation of the component (such as with a peroxide, sodium hypochlorite or ozone), reduction of the component, degradation or hydrolysis of the component and solubilisation of the component. A specific nonlimiting example is the addition of HCl to a fluid comprising Fe(III)OOH to produce iron chloride and water.

In certain embodiments, the clarifying agent is a solvent to solubilize a component of the opaque fluid which causes the fluid to be opaque, thereby clarifying the fluid to enable distinguishing between particulate matter in the fluid and the fluid itself. For example, the addition of an organic solvent to a vial containing an opaque injectable pharmaceutical suspension may help solubilize an otherwise insoluble component and thus clarify the fluid to enable the inspection of the fluid for the presence or absence of particulate matter, or enable the detection, identification and/or visualization of particulate matter therein. In certain embodiments, the solvent is an organic solvent. In certain embodiments, the solvent is an inorganic solvent. In certain embodiments the clarifying agent is an aqueous solvent. In certain embodiments, the clarifying agent is water. In certain embodiments, the solvent is selected from the group consisting of methylene chloride, chloroform, dimethyl sulfoxide (DMSO), dimethylformamide (DMF), dimethylacetamide (DMac) and tetrahydrofuran. In certain embodiments, the opaque fluid treated with the solvent is a suspension. In certain embodiments, the opaque fluid treated with the solvent is a solution. In certain embodiments, the solution is a colloidal solution.

In certain embodiments, the clarifying agent is an acid or base. For example, the addition of HCl 12N or NaOH 10N to a vial containing an opaque solution, for example an injectable pharmaceutical, may help solubilize the otherwise insoluble component and thus clarify the fluid to enable distinguishing between particulate matter in the fluid and the fluid itself, to enable the inspection of the fluid for the presence or absence of particulate matter, or enable the detection, identification and/or visualization of particulate matter therein. In certain embodiments, the clarifying agent is an acid. In certain embodiments, the acid is selected from the group consisting of hydrochloric acid, sulfuric acid, nitric acid, acetic acid and phosphoric acid. In certain embodiments, the acid is HCl. In certain embodiments, the acid is 12N HCl. In certain embodiments, the clarifying agent is a base. In certain embodiments, the base is NaOH. In certain embodiment, the base is 10N NaOH. A specific non-limiting example is the addition of 12N HCl to an iron oxide solution (Fe III) to transform the iron oxide solution (Fe III) (dark red opaque solution) to an iron chloride (Fe II) solution (light orange solution). This simple phenomenon allows for a suitable clarification of the solution to enable distinguishing between particulate matter in the fluid and the fluid itself, to enable the inspection of the fluid for the presence or absence of particulate matter, or enable the detection, identification and/or visualization of a particulate matter therein. In certain embodiments, the opaque fluid treated with an acid or base is a suspension. In certain embodiments, the opaque fluid treated with an acid or base is a solution. In certain embodiments, the solution is a colloidal solution.

In certain embodiments, the clarifying agent is a gas, for example, ozone.

In certain embodiments, the clarifying agent is selected from the group consisting of an acid, a base, an oxidizing agent, a reducing agent, a degradation agent, a hydrolytic agent, and a solvent.

As will be understood by those skilled in the art, the selection of an appropriate agent will be dependent on the type of fluid being tested and the formulation components of the fluid to be eliminated or modified to enable clarification. As will also be understood by those skilled in the art an appropriate clarifying agent will be one that does not affect particulate matter in the fluid such that the particulate matter is not distinguishable in the clarified fluid.

In another embodiment of the invention, the invention relates to a device for distinguishing between particulate matter in an opaque fluid and the fluid itself, the device comprising means for clarifying the fluid to enable distinguishing between the particulate matter in the fluid and the fluid itself.

In another embodiment of the invention, the invention relates to a device for detecting the presence or absence of particulate matter in an opaque fluid, the device comprising means for clarifying the fluid to enable detecting the presence or absence of particulate matter therein.

In another embodiment of the invention, the invention relates to a device for visualizing the presence or absence of particulate matter in an opaque fluid, the device comprising means for clarifying the fluid to enable visualizing the presence or absence of particulate matter therein.

In another embodiment of the invention, the invention relates to a device for inspection of an opaque fluid for the presence or absence of particulate matter, the device comprising means for clarifying the fluid to enable inspection of the fluid for the presence or absence of particulate matter therein.

In another embodiment of the invention, the invention relates to a device for identification of particulate matter in an opaque fluid, the device comprising means for clarifying the fluid to enable identification of the particulate matter therein.

The term “device” is intended to mean an instrument or tool capable of enabling the inspection of a fluid for the presence or absence of particulate matter, or enabling the detection, identification and/or visualization of particulate matter in a fluid. In certain embodiments, the device is manually operated. In certain embodiments, the device is automated. Such devices are well known in the art. In certain embodiments, the device is, or comprises, a microscope. In certain embodiments, the device is, or comprises, a light box. In certain embodiments, the device is, or comprises, a camera. In certain embodiments, the device is a particle counter, such as are well known in the art. In certain embodiments, the particle counter is selected from the group consisting of HIAC/ROYCO, Accusizer and FPIA systems.

The term “means for clarifying the fluid” is intended to mean an apparatus, system, process or method for adding to an opaque fluid an appropriate clarifying agent selected to eliminate or modify a component of the opaque fluid such that the component no longer makes the fluid opaque. Apparatuses, systems, processes or methods for adding an agent, such as a liquid agent, to a container containing a fluid are well known in the art. In certain embodiments, the means comprises a needle, a spout or a tip (such as a needle operationally connected to a syringe, or a pipette tip operationally connected to a pipette). In certain embodiments, the means comprises a needle. In certain embodiments, the needle penetrates a container containing the fluid, or a container closure, to enable adding the agent to the fluid therein. In certain embodiments, the container closure is a cap or stopper, In certain embodiments, the needle is operationally connected to a syringe, or other measuring or dispensing system, such as a pump and reservoir for the agent. In certain embodiments, the container closure is opened or removed from the container to enable adding the agent directly into the fluid therein, without need for penetrating the container or container closure. In certain embodiments, the means comprises a pipette tip. In certain embodiments, the pipette tip is operationally connected to a pipette, or other measuring or dispensing system, such as a pump and reservoir for the agent. In certain embodiments adding the agent to the fluid is performed manually. In certain embodiments adding the agent to the fluid is automated.

The term “automated” is intended to include semi-automated and fully automated.

The term “animal” tended to mean both human and non-human. In certain embodiments the animal is a human. In certain embodiments the animal a non-human, for example a mammal, for example a domesticated mammal. In certain embodiments, the animal is selected from the group consisting of dog, cat, horse swine, cattle, sheep and goat.

In another embodiment of the invention, the invention relates to a quality control process for release of a quantity of opaque fluid during or after manufacture of the fluid, comprising a use, a method, use of a method, and/or device described herein.

In certain embodiments, clarifying the fluid comprises removing a component of the opaque fluid which causes the fluid to be opaque, thereby clarifying the fluid.

For any types of quality control purposes, sufficient numbers of samples are collected and tested to meet appropriate quality criteria. In certain embodiments the criteria are industry standard AQL criteria.

In certain embodiments a product to be tested for the presence or absence of particulate matter is not an opaque fluid but is instead a dry solid, such as a powder or lyophilisate. The same disclosure as encompassed herein for fluids is applicable to testing a dry solid for particulate matter. In the case of a dry solid, the dry solid is first converted to a fluid format. As will be understood by those skilled in the art, the selection of an appropriate method to convert a dry solid to a fluid format will be dependent on the dry solid being tested. In certain embodiments the fluid so formed is an opaque fluid, which can be tested for particulate matter as described herein. In certain embodiments, the fluid so formed is transparent to a sufficient degree to directly enable inspection of the solid for the presence or absence of particulate matter, or enable the detection, identification and/or visualization of a particulate matter therein. In certain embodiments, converting the dry solid to a fluid format comprises treating the dry solid with a solvent (for example, an aqueous solvent, an inorganic solvent, or an organic solvent) to help solubilize the dry solid. In certain embodiments, the solvent is an aqueous solvent, such as water. In certain embodiments, the solvent is an organic solvent. In certain embodiments, the solvent may include a solubilization enhancing agent.

The term “solubilization enhancing agent” is intended to mean any agent that aids in the solubilization of a dry solid. Such agents will be well known to those of skill in the art and include, but are not limited to, pH modifiers, water-soluble organic solvents, water-insoluble organic solvents, surfactants, triglycerides, cyclodextrins and phospholipids.

In certain embodiments, the dry solid comprises an compound. In certain embodiments the iron compound is an iron carbohydrate complex.

In certain embodiments, the dry solid is a cosmetic product.

In certain embodiments, the opaque fluid or dry solid is a pharmaceutical composition. In certain embodiments, the pharmaceutical composition is for administration via a method well known to those skilled in the art including, but not limited to, oral, transdermal, inhalation, nasal, topical, intravaginal, ophthalmic, intraaural, intracerebral, rectal, sublingual, buccal, intraurethral, and parenteral, including injectable such as intravenous, intra-arterial, intramuscular, and subcutaneous. In certain embodiments, the pharmaceutical composition is for parenteral administration. In certain embodiments, the pharmaceutical composition is for injection or intravenous administration.

### EXAMPLES

The following examples are for illustrative purposes only and are not intended to limit the scope of the invention.

### Example 1

### Testing of Packaged Vials of Iron Dextran Solution

Three batches of iron dextran solution in 5 mL vials and three batches of iron dextran solation in 2 mL vials were tested for particulate matter. Following AQL S4 criteria, 32 vials (4%) were required for testing per batch. The limit for batch rejection was 3 vial displaying particulate matter. Three to four spiked s mole controls were tested per batch. In test studies, with vials spiked with glass beads (100 microns), metal, hair and paper fiber, no particle degradation was observed as a result of treating the solutions with HCl. The results are shown in Table 2. All spiked sample controls tested positive for particulate matter.

The iron dextran formulation is shown in Table 1.

TABLE 1

| | Amount | |
|---|---|---|
| Component | mg | % |
| Iron dextran | 50 (as | 5.0 |

| | |
|---|---|
| Hydrochloric acid | pH adjustment |
| Sodium hydroxide | pH adjustment |
| Water for injection | q.s. 1 mL |
| pH | 5.2-6.5 |

*Corresponding to 312.5 mg of iron (III)-hydroxide dextran complex

### a. 5 mL Vial

Fifty individual 5 mL vials containing iron dextran solution are collected for testing. The vials are left in an upright position for 12-24 hours, after which the lids are removed. Approximately 1.5 mL of 12N HCl is added to each vial using a 3 mL syringe having a 27 gauge (½) needle, The vials are inverted ten times and then placed in a water bath set at 60° C. for 45 minutes. After the bath, the vials are removed, dried, inverted again 10 times and are ready for visual inspection. Prior to each visual inspection, the vials are inverted 10 times.

### b. 2 Vial

Fifty individual 2 mL vials containing iron dextran solution are collected. The vials are in an upright position for 12-24 hours, after which the vial lids are removed. As the headspace volume in the 2 mL vial is limited, approximately 0.2 mL of iron dextran solution is removed using a 3 mL syringe having a 30 gauge needle. Approximately 0.6 mL of 12N HCl is added to each vial using a 3 mL syringe having a 27 gauge (112) needle, The vials are inverted ten times and then placed in a water bath set at 60√ C. for 45 minutes. After the bath, the vials are removed, dried, inverted again 10 times and are ready for visual inspection. Prior to each visual inspection, the vials are inverted 10 times.

TABLE 2
Real Case Study: Iron Dextran Injectable Soluton

| | Inspection | | |
|---|---|---|---|
| | | Number of vials | Batch |
| | | found with | Quality |
| | | particulate matter | Control |
| Lot | Volume | (non-control vials) | Results |
| A | 5 mL | 0 | Pass |
| B | 5 mL | 3 | Pass |
| C | 5 mL | 2 | Pass |
| D | 2 mL | 5 | Fail |
| E | 2 mL | 3 | Pass |
| F | 2 mL | 2 | Pass |

### Example 2

### Testing of Packaged Vials of Medroxyprogesterone Acetate Injection Suspension

In test studies with vial spiked with glass beads (100 mirons) or hair, no particle degradation was observed as result of treating the suspension with methylene chloride or chloroform.

One batch of Medroxyprogesterone Acetate injection suspension in 2 mL vials, in addition to spiked sample controls, was tested for particulate matter.

The Medroxyprogesterone Acetate injection suspension formulation is shown in Table 3.

TABLE 3

| | Amount | |
|---|---|---|
| Component | mg | % |
| Medroxyprogesterone Acetate | 150 | 15.0 |
| Polyethylene glycol 3350 | 28.9 | 2.89 |
| Polysorbate 80 | 2.41 | 0.24 |
| Sodium Chloride | 8.68 | 0.868 |
| Methyl paraben | 1.37 | 0.137 |
| Propyl paraben | 0.150 | 0.015 |

| | |
|---|---|
| Hydrochloric acid | pH adjustment |
| Sodium hydroxide | pH adjustment |
| Water for injection | q.s. 1 mL |

Three 2 mL vials containing Medroxyprogesterone Acetate injection suspension are collected. Approximately 1.0 mL of methylene chloride or 1.0 mL of chloroform is added to each vial using a 3 syringe having a 27 gauge (½) needle. The vials are inverted ten times and vortexed for 2 minutes. After the mixing step, the vials are removed and kept upright for 24 hours. Then, vials are ready for visual inspection. Prior to each visual inspection, the vials are inverted 10 times. All spiked sample controls tested positive for particulate matter.

Although various embodiments of the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments may be interchanged either in whole or in part. Therefore, the spirit and scope of the appended claims should not be limited to the description of the preferred versions contained therein.

What is claimed is:

1. A method for distinguishing between particulate matter in an opaque fluid and the opaque fluid itself, the method comprising clarifying the fluid wherein clarifying the fluid enables distinguishing between the particulate matter and the fluid.

2. The method of claim 1, wherein clarifying the fluid comprises removing a component of the fluid which causes the fluid to be opaque.

3. The method of claim 2 wherein removing a component of the fluid which causes the fluid to be opaque comprises treating the fluid with an agent selected from the group consisting of an acid, a base, an oxidizing agent, a reducing agent, a degradation agent, a hydrolytic agent, and a solvent.

4. The method of claim 1, wherein the fluid is selected from the group consisting of solution, suspension, emulsion, gel, cream and paste.

5. The method of claim 4, wherein the fluid is selected from the group consisting of solution, emulsion and suspension.

6. The method of claim 1, wherein the fluid is for a use selected from the group consisting of pharmaceutical, cosmetic and consumption.

7. The method of claim 6, wherein the fluid is a pharmaceutical fluid.

8. A method for detection of the presence or absence of particulate matter in an opaque fluid, the method comprising clarifying the fluid to enable detection of the presence or absence of particulate matter therein.

9. The method of claim 8, wherein clarifying the fluid comprises removing a component of the fluid which causes the fluid to be opaque.

10. The method of claim 9 wherein removing a component of the fluid which causes the fluid to be opaque comprises treating the fluid with an agent selected from the group consisting of an acid, a base, an oxidizing agent a reducing agent, a degradation agent, a hydrolytic agent, and a solvent.

11. The method of claim 8, wherein the fluid is selected from the group consisting of solution, suspension, emulsion, gel, cream and paste.

12. The method of claim 11 wherein the fluid is selected from the group consisting of solution, emulsion and suspension.

13. The method of claim 8, wherein the fluid is for a use selected from the group consisting of pharmaceutical cosmetic and consumption.

14. The method of claim 13, wherein the fluid is a pharmaceutical fluid.

15. A method for inspection of an opaque fluid for the presence or absence of particulate matter, the method comprising clarifying the fluid to enable the inspection of the fluid for the presence or absence of particulate matter therein.

16. The method of claim 15, wherein clarifying the fluid comprises removing a component of the fluid which causes the fluid to be opaque.

17. The method of claim 16 wherein removing a component of the fluid which causes the fluid to be opaque comprises treating the fluid with an agent selected from the group consisting of an acid, a base, an oxidizing agent, a reducing agent, a degradation agent, a hydrolytic agent, and a solvent.

18. The method of claim 15, wherein the fluid is selected from the group consisting of solution, suspension, emulsion, gel, cream and paste.

19. The method of claim 18, wherein the fluid is selected from the group consisting of solution, emulsion and suspension.

20. The method of claim 15, wherein the fluid is for a use selected from the group consisting of pharmaceutical, cosmetic and consumption.

21. The method of claim 20, wherein the fluid is a pharmaceutical fluid.
